(19) United States
(12) Patent Application Publication
(10) Pub. No.: US 2015/0289979 A1
(43) Pub. Date: Oct. 15, 2015

(54) BONE IMPLANT MADE OF AT LEAST TWO DIFFERENT ABSORBABLE AND BIODEGRADABLE MATERIALS ADAPTED TO BE COMBINED AS HYBRID OR COMPOSITE MATERIAL

(71) Applicant: KARL LEIBINGER MEDIZINTECHNIK GMBH & CO. KG, Muehlheim (DE)

(72) Inventors: Lorenz GABELE, Muehlheim (DE); Frank REINAUER, Muehlheim (DE); Tobias WOLFRAM, Muehlheim (DE); Wolfgang MUELLER, Muehlheim (DE)

(21) Appl. No.: 14/441,242

(22) PCT Filed: Nov. 11, 2013
(86) PCT No.: PCT/EP2013/073507

§ 371 Date: May 7, 2015

(30) Foreign Application Priority Data
Nov. 9, 2012 (EP) 12192006.0

(51) Int. Cl. A61F 2/28; A61L 27/58; A61L 27/18; A61L 27/04

(52) U.S. Cl. CPC A61F 2/28; A61L 27/04; A61L 27/58; A61L 27/042; A61L 27/047; A61L 27/18; A61F 2002/2835; A61F 2002/30032

(57) ABSTRACT

A bone implant includes a support structure made of metal ahoy and includes a biodegradable and absorbable protective structure, the protective structure being arranged at and/or on the support structure so that the support structure is protected from contacting any aggressive body fluid in a position anchored in a bone of an individual such as a mammal, wherein the support structure is surrounded by and interspersed with the protective structure.

### BACKGROUND OF THE INVENTION

The invention relates to a bone implant comprising a support structure made of a biodegradable metal or a biodegradable metal alloy and comprising a biodegradable and absorbable protective structure, the protective structure being arranged at and/or on the support structure so as to protect the support structure from contacting an aggressive body fluid in a position anchored in a bone of an individual.

From the state of the art absorbable biodegradable materials such as poly-D,L-lactic acid (PDLLA), polyglycollic acid (PGA) or polycaprolactone (PCL) are known already. However, these materials have definitely lower mechanical strength properties than standard metallic materials such as titanium or implant steel.

Biodegradable ceramic implant materials unfortunately in many fields exhibit insufficient breaking and reverse bending strength and frequently are difficult to model or not at all adapted to be “chair-side” modeled.

Although the use of magnesium and magnesium alloys in the domain of implants is basically known, especially as they permit high strengths, the absorption takes place only under sub-optimal conditions, however, in terms of kinetics, physiological aspects, gas development, degradation mechanisms and degradation products, when used at the bone of a mammal and especially of human beings. Frequently absorption is carried out too quickly.

It is the object of the present invention to eliminate the drawbacks known from the state of the art and to provide an optimally absorbing bone implant which has a sufficiently high strength at any instant of bone regeneration. In addition, a bone implant of this type is intended to be cost-efficient and to allow an as long storage time as possible without deterioration of the desired properties.

The compatibility of the material with a human bone and the tissue surrounding the human bone is to be ensured.

### SUMMARY OF THE INVENTION

According to the invention, this is achieved in a generic bone implant in that the support structure is surrounded by and/or interspersed with the protective structure.

In this way at least two or even more biodegradable materials, possibly combined with non-degradable contents or active substances, having different properties are used for adjusting the different chemical, physical, mechanical and biological properties of the overall system in a well-defined manner.

The implant exhibits high strength when e.g. magnesium or magnesium alloys are used. With previously known magnesium or magnesium alloys the degradation then occurred too quickly for the important clinical applications at the bone. If the bone implant is made, according to the invention, of magnesium or a magnesium alloy in combination with a protective structure which, although possibly exhibiting low strength, ensures optimum degradation kinetics, an improved design becomes possible. With the combination of the two or more surrounding or interspersing materials according to the invention, the support structure can be protected against too rapid degradation. Thus it is possible to adjust the degradation dynamics/kinetics. An adjustment of the physiological/metabolic activity in the environment of the implant can be specifically performed. It is also possible to adjust chemical conditions such as pH value, concentration of the degradation products etc. in response to demand. It becomes possible to adjust the mechanical properties of the original implant and the implant properties during the degradation phase. During the state of implant it is ensured that high mechanical strength is given due to the structure of the higher-strength, preferably metallic component. Although the higher-strength component absorbs, the enveloping structure of the low-strength component is retained for a longer time, for example, and protects the higher-strength component from a possibly too rapid absorption. The protective sheathing of the higher-strength material by means of the low-strength component results, e.g., in a mechanical protective function for the surrounding tissue when the absorption kinetics is appropriately adjusted. A reasonable way of tissue protection can be realized.

Advantageous embodiments will be illustrated hereinafter.

It is beneficial when the metal alloy is a biodegradable and absorbable material, because in such case also the support structure is completely degradable by the body.

It is also beneficial when the protective structure is made of non-metallic material or includes (contains) the same.

When the material includes at least one element or plural elements of the group consisting of magnesium, iron, zinc, strontium, fluorine, manganese and calcium as well as possible ions thereof, especially suited materials and material alloys can be employed.

It is useful when the protective structure includes a material different from the support structure or is made of such different material. The different properties then can be combined with each other in line with demand.

An advantageous embodiment is also characterized in that the protective structure is made of plastic material and preferably includes polylactide compounds. The polylactide compounds ensure absorption kinetics optimum in terms of time.

It has also turned out to be of advantage when the protective structure includes polylactic acid such as PLA, and/or polyglycollic acid (PGA) and/or polycaprolactone (PCL) such as poly-ε-caprolactone-co-lactide, PDLLA-TCP, PDLLA-calcium carbonate and/or poly-D-lactic acid (PDLA).

In order to obtain an especially advantageous embodiment it is of advantage when the support structure and the protective structure are penetrated by or mixed with each other in the way of a material composite component or a hybrid component, e.g. in the way of single-sized concrete. The material composite exhibits constant degradation characteristics in its entire 3D volume so that it desensitizes the bone implant against mechanical surface impairment, e.g. during contouring, bending, separating and/or mechanical machining. The drawbacks as they occur e.g. during so called “coating” of implants without material composite can thus be avoided. Frequently during mechanical machining of the implant, e.g. during contouring or during separating, the coating is injured, i.e. the locally occurring “corrosive” attack then results in undesired degradation kinetics of the implant at such site, which is avoided by the porous configuration according to the invention. Also during bending the protective layer is prevented from being injured, i.e. the magnesium implant located there beneath is prevented from being exposed and exhibited to corrosion, possibly even at highly loaded cross-sections.

It is further beneficial when the support structure includes a shape of adjacent supporting particles such as grains and/or balls and/or lattice elements such as bars and/or hexagonal elements and/or triangles and/or crow's foot elements and/or honeycombs and/or fibers. In such case especially loadable and versatile bone implants can be generated.

When the support structure has the geometry of metal foam, manufacture can be facilitated. It is also advantageous when the supporting particles are adjacent to one another so that cavities are provided there between. This facilitates introduction of the protective structure.

If at least one cavity or preferably a plurality of cavities, for example all cavities, are filled at least partially, preferably completely with the material of the protective structure, a compact bone implant of especially high strength can be obtained so that the bone implant remains protected against corrosion in accordance with the degradation kinetics adjusted according to the invention.

It is especially expedient when some of the cavities are completely filled with material of the protective structure and some of the cavities are filled only partially with material of the protective structure, and preferably the ratio of completely filled cavities to partially filled cavities is 20:1 to 10:1, further preferably the ratio is approximately 15:1.

During tests it has turned out to be especially expedient when the support structure and the protective structure are entangled and/or combined with each other in sandwich construction.

For reasonably combining the individual properties it is advantageous when the support structure exhibits higher strength than the protective structure.

Furthermore, it is advantageous when the bone implant is in the form of a plate, a cranial bone implant, a nail, a lattice, a fabric or a screw. A rivet form can be chosen as well.

It is moreover advantageous when the support structure is in the form of a sintered structure. It is possible that the individual composite parts such as magnesium pellets are “welded” to one another at their contact points to produce a sintered structure. The free space inside the composite is filled e.g. with PDLLA and prevents too rapid corrosive attack of the body fluids on the magnesium support structure. The pellets can also be replaced by standard geometries such as three-dimensional triangles of honeycomb layers in the way of an armor barrier geometry or lattice structures. Furthermore structures in which the support structure is made e.g. in the form of the afore-mentioned standard geometries in a sintered structure are advantageous.

The layers can be combined of metal and PDLLA in sandwich construction. The metal foam can also be filled with PDLLA. Basically also solid implants made of metal with surrounding PDLLA material are possible, unless such systems are intended to be mechanically machined.

When the protective structure is provided on the support structure to be incomparably thicker in areas of higher mechanical and/or chemical load than in neighboring areas, also in the case of highly loaded bone implants an absorption process optimally controlled in time can be ensured.

It is useful when the bone implant is not designed to be hollow or flexible like an endoluminal vascular prosthesis.

Hereinafter the invention will be illustrated in detail with the aid of a drawing. The different embodiments are visualized in the figures and will be explained in detail hereinafter.

### BRIEF DESCRIPTION OF THE DRAWINGS

FIG. 1 shows a schematic view of a composite starting material of which the bone implant is made and in which the support structure is provided in spherical shape,

FIG. 2 shows a schematic view of a composite starting material of which the bone implant is made and in which the support structure is provided in fiber or plate-shaped form,

FIG. 3 shows a schematic view of a composite starting material of which the bone implant is made and in which the support structure is provided in bar-shaped form,

FIG. 4 shows a schematic view of a composite starting material of which the bone implant is made and in which the support structure is provided in a first type of bracings,

FIG. 5 shows a schematic view of a composite starting material of which the bone implant is made and in which the support structure is provided in a second type of bracings,

FIG. 6 shows a schematic view of a composite starting material of which the bone implant is made and in which the support structure is provided in honeycomb form,

FIG. 7 shows a schematic view of a composite starting material of which the bone implant is made and in which the support structure is provided in the form of an armor barrier, and

FIG. 8 shows a schematic view of a composite starting material of which the bone implant is made and in which the support structure is in lattice form.

The figures are merely schematic and only serve for the comprehension of the invention.

### DETAILED DESCRIPTION OF THE INVENTION

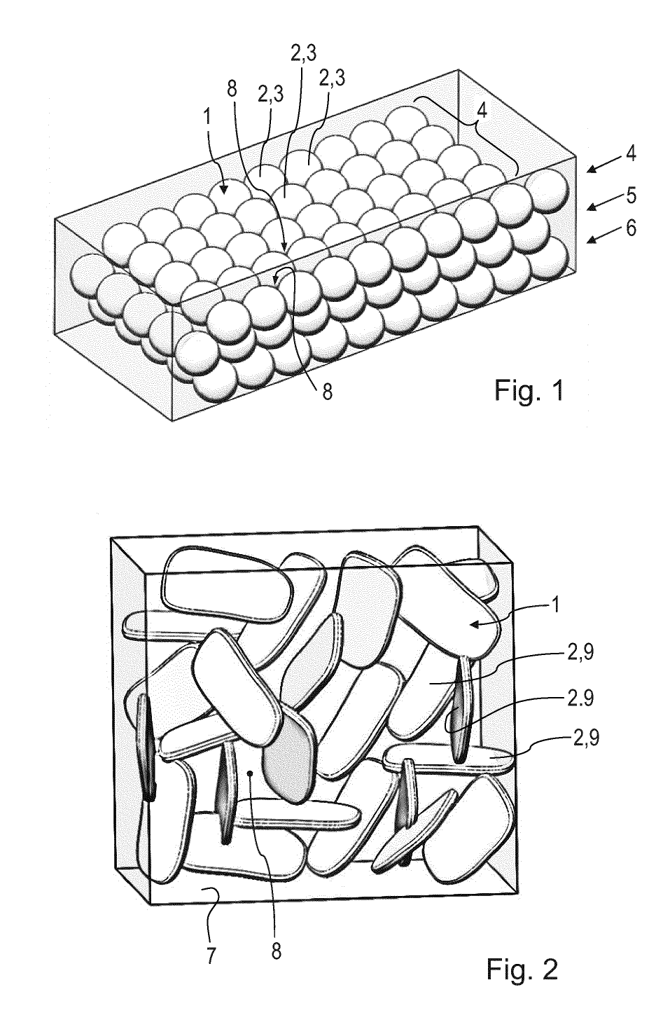

In FIG. 1 a starting material for a bone implant is shown, wherein the specific shape of the bone implant is not reproduced. It is possible that such bone implant has the final shape of a plate, a cranium implant, a nail, a lattice, a fabric, a rivet or a screw. Cutting and non-cutting forming methods can be employed to obtain the final shape of the implant.

The starting material of which the bone implant according to the invention is made includes in the embodiment shown in FIG. 1 a support structure 1 comprising support elements 2 having a spherical shape. The support elements 2 thus have the form of balls 3.

Plural balls 3 are contacting one other and are arranged in one or more layers, for example a first layer 4. There are further balls 3 in further layers. In the embodiment shown here a further second layer of balls 3 and a further third layer of balls 3 is provided. While the first layer is provided with reference numeral 4, the second layer is provided with reference numeral 5 and the third layer is provided with reference numeral 6.

A ball 3 of the second layer contacts four balls 3 of the same layer, unless it is located at the margin of the layer. At least one ball 3 of the first layer 4 and one ball 3 of the third layer 6 equally contact said ball 3 of the second layer 5.

In the shown embodiment the balls 3 of the individual layers are arranged in the way of a close spherical packing, however. Thus hexagonal spherical layers are provided. Apart from such a hexagonal close spherical packing, also a cubically close spherical packing is possible. Also the “dhcp” type of structure is possible. As an alternative, it is also possible, as a matter of course, that the individual balls 3 are arranged in the way of a lattice cubically centered in space (bcc).

All balls 3, i.e. all balls 3 of all different layers of supporting elements 2, are surrounded by a biodegradable and/or bioabsorbable protective structure 7. The protective structure 7 has equally penetrated cavities 8 between the individual spherical support elements 2 so that the balls 3 are differently retained in the protective structure 7.

The balls 3 which are communicated with other balls 3, i.e. the support elements 2 contacting one other, are adhesively bonded, for example welded, to each other. In the present case a sintering method was employed to interconnect the support elements 2. It has turned out to be especially efficient when a laser acts on the material of the support elements 2 provided in powder form.

The path for corrosive material is extended by appropriately attaching the supporting elements 2 to each other. The corrosive material which enters into the interior through a fracture in the protective structure 7, for instance, has to cover an especially long distance from one support element 2 susceptible to the corrosive attack to the next so as to be able to attack said further support element 2 at all. This has effects on the duration of corrosion. It takes especially long until the next support element 2 has been attacked and degraded.

As a consequence, it takes especially long until the support structure 1 is weakened and all support elements 2 are degraded some time. In this way the degrading kinetics can be specifically adjusted by means of the arrangement of the individual support elements 2 inside the protective structure 7. The degrading kinetics is dependent on the respective use of material in the support structure 1 and the protective structure 7. The materials employed in this respect can be selected in a well-targeted manner and adjusted to one other for the desired purpose.

Also the embodiments shown in FIGS. 2 to 8 follow this principle. There, too, the individual support elements are always interconnected, preferably adhesively bonded, further preferably welded or connected by sintering. The finished bone implant is surrounded by the protective structure 7 preferably on all sides.

The support elements 2 of the support structure 1 used in the embodiments of FIGS. 2 to 8 exhibit different forms.

The support elements 2 of the embodiment according to FIG. 2 are in the form of fibers 9 or plates, respectively. The fibers 9 are planar plate-shaped structures that can also be partly intersected. They can also be filaments though. At their margins they have roundings, but they can as well be rectangular or can even be tapered. It is also possible that the fibers themselves are in the form of plates and in such case are not planar but have an undulated form, for example a convex or concave form. They can have a constant thickness, but they may also be ellipsoidal. In particular a lens shape is possible.

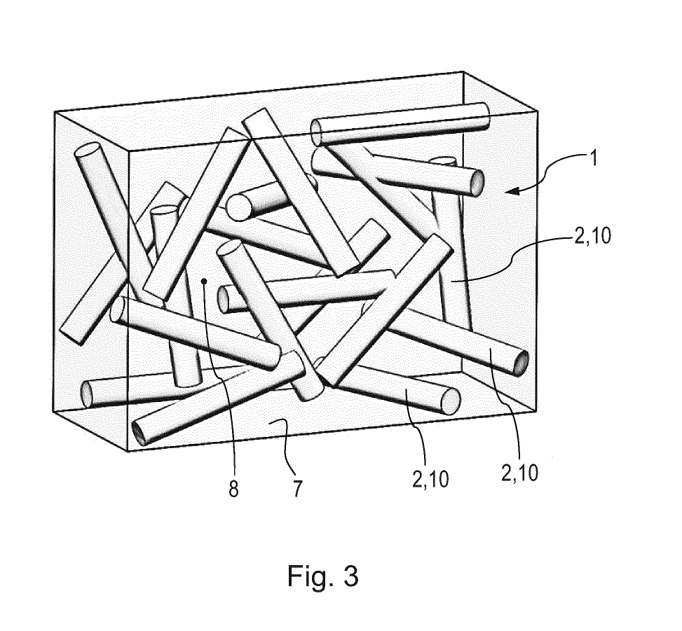

In the embodiment according to FIG. 3 the supporting elements 2 are in the form of bars. The bars are provided with the reference numeral 10 and are column-shaped. They have a circular cross-section, but they can as well have a polygonal cross-section. The bars 10 have a constant cross-section. The cross-section can also vary, however.

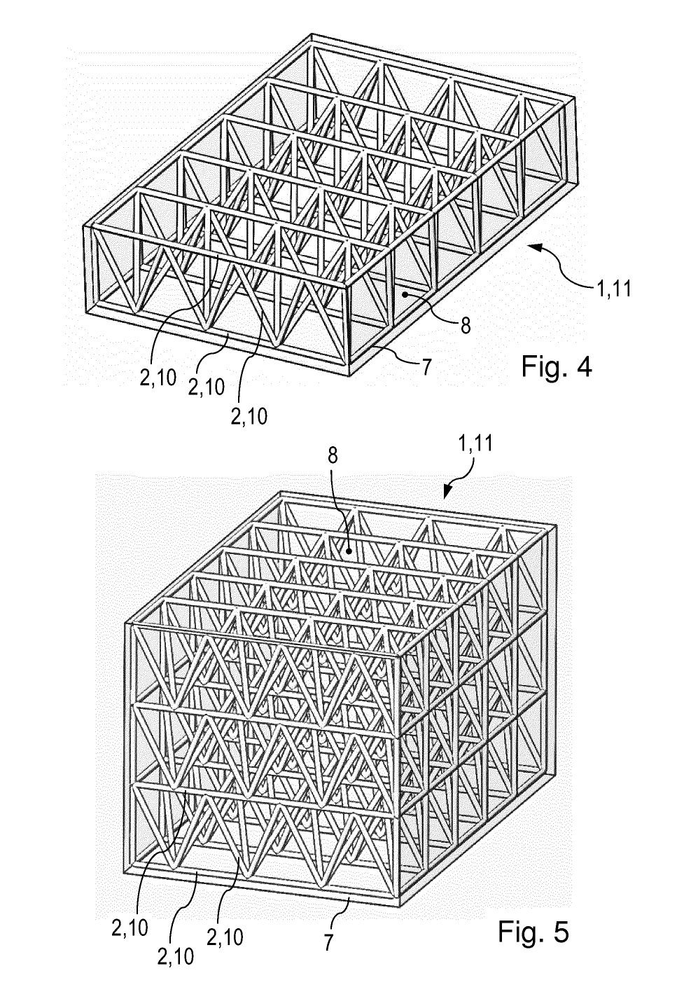

At least one bar 10 contacts a further bar 10 and is adhesively bonded in the area of contact, as already explained concerning the embodiments of FIGS. 1 and 2. It has turned out to be advantageous when 2, 3, 4, 5, 6, 7, 8, 9, 10, 11 and more bars are interconnected at a time. In this case, too, as already in the two afore-described embodiments, the protective structure 7 is interspersed with the support structure 1 and surrounds the latter, respectively.

Whereas in the embodiment according to FIG. 3 the individual bars 10 are provided almost randomly inside the protective structure 7, in the embodiment of FIGS. 4 and 5 the bars 10 are positioned in the way of bracings 11 of a geometrically recurring arrangement. The bracing 11 can also be designed to be point-symmetric.

The embodiments of FIGS. 4 and 5 differ by the number of layers of bracings 11. In the embodiment according to FIG. 5 three layers of bracings 11 are provided, whereas in the embodiment according to FIG. 4 only one layer of bracings 11 is provided.

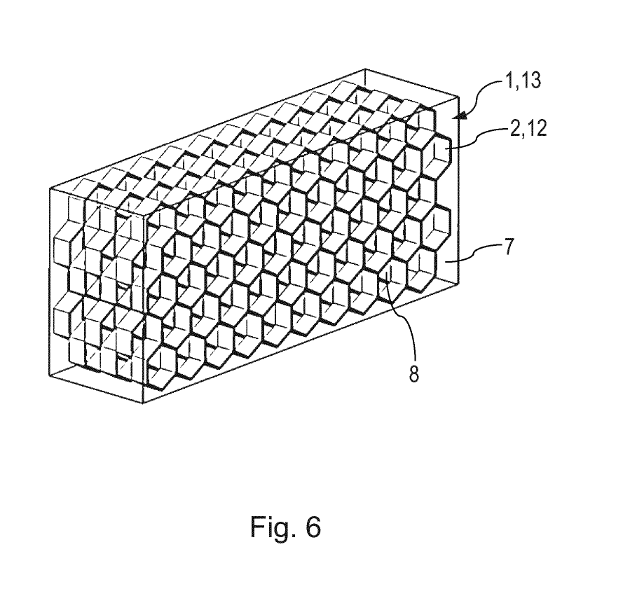

In the embodiment according to FIG. 6 the individual support elements 2 are designed in the way of plates 12, the individual plates 12 forming layers of a honeycomb-type structure, wherein individual layers of the honeycomb-type structure are offset with respect to each other. The individual plates 12 thus form a complex honeycomb structure 13.

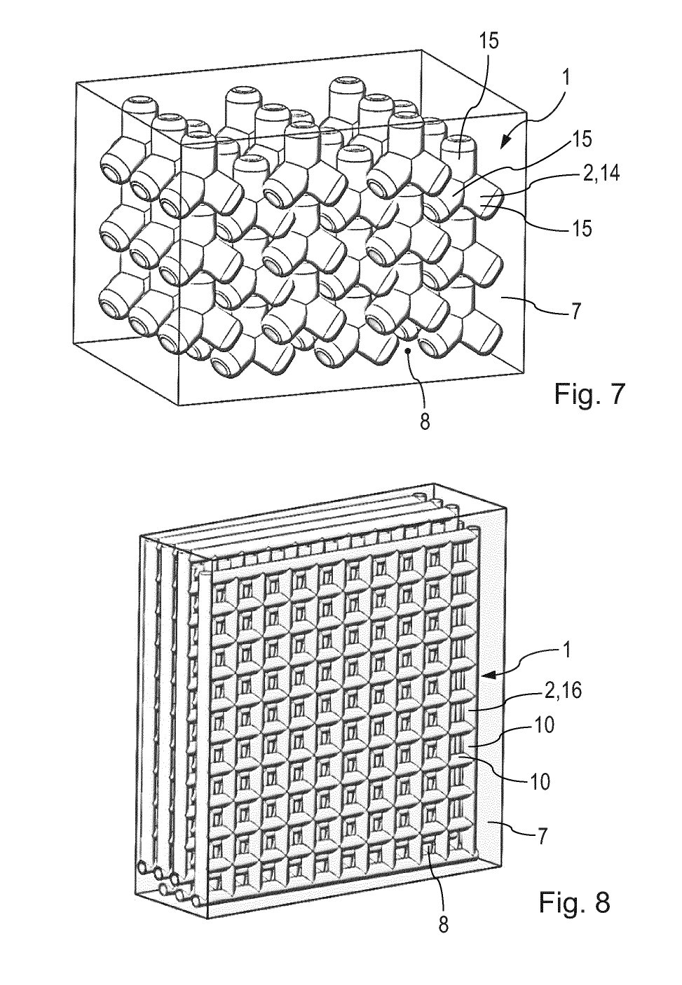

In FIG. 7 the support structure 1 is composed of individual support elements 2 formed in the way of armor barrier elements 14. Each armor barrier element 14 includes four cylindrical segments 15. Each of the four cylindrical segments 15 has the same solid angle from the closest cylindrical segment 15 of the same armor barrier element 14. The cylindrical segments 15 are rounded at the ends, but they can also have edges that are not rounded. The individual cylindrical segments 15 can be made of solid matter, but they can as well be hollow.

The cavities 8 are filled in turn by the protective structure 7.

The individual cylindrical segments 15 can have a constant diameter or a variable diameter. The individual cylindrical segments 15 can have the same diameter or the same diametrical course as the neighboring cylindrical segments or can have a diameter or diametrical course different therefrom.

In FIG. 8 another embodiment of a support structure 1 according to the invention is shown in a bone implant to be produced according to the invention. The individual support elements 2 of the support structure 1 are combined with each other in the way of a lattice 16 preferably in different layers. The individual bars 10 of the lattice 16 are orthogonally intersecting. In order to achieve an as high mechanical strength as possible, especially after implantation, it is advantageous when the individual support elements are provided in the protective structure 7 as closely adjacent to each other as possible.

Basically a configuration similar to a single-sized concrete is also possible.

1.-15. (canceled)

16. A bone implant comprising a support structure made of a metal alloy and comprising a biodegradable and absorbable protective structure, wherein
the protective structure comprises a material different from the support structure and the support structure is surrounded by the protective structure,
the support structure comprises a plurality of individual interconnected support elements, and
the protective structure is arranged at and/or on the support structure so that the implant is surrounded by the protective structure on all sides and the support structure is protected from contacting any aggressive body fluid when the bone implant is anchored in a bone of a human or other mammal.

17. The bone implant according to claim 16, wherein the metal alloy is a biodegradable and absorbable material.

18. The bone implant according to claim 16, wherein the alloy of the support structure includes at least one element and, optionally, ions thereof of the group consisting of magnesium, iron, zinc, strontium, fluorine, manganese and calcium.

19. The bone implant according to claim 16, wherein the protective structure is non-metallic.

20. The bone implant according to claim 16, wherein the support structure and the protective structure are penetrated by or mixed with each other in a form of a material composite component or a hybrid component.

21. The hone implant according to claim 20, wherein the support elements of the support structure are in a form of at least one of mutually adjacent supporting particles or lattice elements or hexagonal elements or triangles or crow's foot elements or honeycombs or fibers.

22. The bone implant according to claim 21, wherein cavities are provided between the support elements of the support structure.

23. The bone implant according to claim 22, wherein at least one of the cavities contains the material of the protective structure.

24. The bone implant according to claim 23, wherein some of the cavities are filled completely with material of the protective structure and some of the cavities of the cavities are filled only partially with the material of the protective structure.

25. The bone implant according to claim 19, wherein the support structure is entangled or combined in sandwich construction with the protective structure.

26. The bone implant according to claim 16, wherein the support structure exhibits higher strength than the protective structure.

27. The bone implant according to claim 16, wherein the hone implant is in the form of a plate, a cranium implant a nail, a lattice, a fabric, a rivet or a screw.

28. The bone implant according to claim 16, wherein the protection structure consists of the material different from the support structure.

29. The bone implant according to claim 19, wherein the protective structure comprises a plastic material.

30. The bone implant according to claim 29, wherein the plastic material comprises a polylactide compound.

31. The bone implant according to claim 29, wherein the plastic material comprises at member selected from the group consisting of polylactic acid (PLA), polyglycollic acid (PGA), poly-caprolactone (PCL), poly-DL-lactide-tricalcium phosphate (PDLLA-TCP), PDLLA-calcium carbonate and poly-D-lactic acid (PDLA).

32. The bone implant according to claim 31, wherein the poly-caprolactone is poly-ε-caprolactone-co-lactide.

33. The bone implant according to claim 24, wherein a ratio of completely filled cavities to partially filled cavities is 20:1 to 10:1.

34. The bone implant according to claim 33, wherein the ratio of completely filled cavities to partially filled cavities is approximately 15:1.
